(19) United States
(12) Patent Application Publication
(10) Pub. No.: US 2026/0157966 A1
(43) Pub. Date: Jun. 11, 2026

(54) FUSOGENIC LIPOSOMES FOR INTRACELLULAR DELIVERY OF PHOSPHOCREATINE

(71) Applicant: The Government of the United States of America, as represented by the Secretary of the Navy, Arlington, VA (US)

(72) Inventors: Okhil K. Nag, Brandywine, MD (US); Eunkeu Oh, Alexandria, VA (US); James B. Delehanty, Washington, DC (US)

(21) Appl. No.: 19/321,446

(22) Filed: Sep. 8, 2025

Related U.S. Application Data
(60) Provisional application No. 63/692,557, filed on Sep. 9, 2024.

(51) Int. Cl. A61K 9/127; A61K 31/661

(52) U.S. Cl. CPC A61K 9/127; A61K 31/661

(57) ABSTRACT

Described herein is a fusogenic liposomal formulation and method for making same for the intracellular delivery of phosphocreatine (PCr) and the resultant generation of ATP.

### CROSS-REFERENCE TO RELATED APPLICATIONS

This patent application claims priority to U.S. Provisional Patent Application Ser. No. 63/692,557, filed Sep. 9, 2024; which is incorporated by reference herein in its entirety into this disclosure.

### FEDERALLY-SPONSORED RESEARCH AND DEVELOPMENT

The United States Government has ownership rights in this subject disclosure. Licensing inquiries may be directed to Office of Technology Transfer, US Naval Research Laboratory, Code 1004, 4555 Overlook Avenue SW, Washington, DC 20375, USA; +1.202.767.7230; techtran@nrl.navy.mil, referencing NC 211809.

### BACKGROUND OF THE SUBJECT DISCLOSURE

### Field of the Subject Disclosure

The present subject disclosure relates generally to fusogenic liposomes. More particularly, the present subject disclosure relates to fusogenic liposomes for intracellular delivery of phosphocreatine.

### Background of the Subject Disclosure

Cellular physiology requires that a source of energy remain readily available to carry out all of the necessary processes involved in cellular homeostasis. Adenosine triphosphate (ATP) is the cell's source of this energy, and it is used in various cellular processes to maintain cellular energy levels. The energy in ATP is contained in the terminal phosphate group, and the hydrolysis of this bond yields energy in the form of calories and heat, which the cell uses to carry out chemical reactions and modulate protein function. ATP is generated in the mitochondria from the precursor adenosine diphosphate (ADP) by oxidative phosphorylation using the phosphate donor, phosphocreatine (PCr). [1] PCr is generated from creatine (Cr) (produced in the mitochondria or taken up by cells from extracellular sources) and ATP using the enzyme creatine kinase (CK). This ATP regeneration process by PCr typically occurs within seconds of intense muscular or neuronal effort, acting as a quickly accessible reserve of high-energy phosphates for the recycling of ATP in the muscle tissues (FIG. 1).

The delivery to and cellular uptake of PCr for the augmentation of ATP production poses a considerable challenge. This arises for several reasons. First, PCr is negatively-charged and does not readily cross the plasma membrane into the cell. In fact, PCr binds to the polar head groups of membrane phospholipids, resulting in decreased membrane fluidity and the stabilization of the membrane bilayer [2]. This aspect has been taken advantage of to prevent heart damage caused by transient ischemia and hypoxia and during heart surgery. Second, the uptake of PCr into the cell and across the mitochondrial membrane is dependent on PCr and creatine transporters which are dysfunctional in a number of conditions [3,4]. Thus, there is a pressing need for techniques that can deliver PCr to the cellular cytosol without direct interaction of PCr with the plasma membrane.

### SUMMARY OF THE SUBJECT DISCLOSURE

The present subject disclosure relates generally to fusogenic liposomes for intracellular delivery of phosphocreatine.

As discussed above, phosphocreatine (PCr) is an intermediate substrate for the generation of adenosine triphosphate (ATP), the cell's primary energy source that drives cellular processes, including muscle contraction, nerve impulse propagation, and chemical synthesis. The cellular delivery of PCr, however, poses a considerable challenge due to its overall net negative charge. This disclosure describes the development of a fusogenic liposomal formulation for the intracellular delivery of PCr and the resultant generation of ATP.

Liposomes were formulated using a mixture of the phospholipids 1,2-dioleoyl-sn-glycero-3-phosphoethanolamine (DOPE), 1,2-dioleoyl-3-trimethylammonium-propane, chloride salt (DOTAP), and aromatic lipids such as 1,2-dioleoyl-sn-glycero-3-phosphoethanolamine-N-[7-nitro-2-1,3-benzoxadiazol-4-yl (NBD-PE), and 1,2-dioleoyl-sn-glycero-3-phosphoethanolamine-N-[lissamine Rhodamine B sulfonyl] (Rhod-PE). PCr was loaded inside the core of the liposomes during the synthesis of the liposomes. NBD-PE or Rh-PE were added to the lipid mixture to fluorescently track and image the fusion of the liposomes to the mammalian plasma membrane. Cellular delivery of PCr with this DOPE/DOTAP/NBD-PE liposome formulation results in a significant (23%) increase in intracellular ATP production.

The following references, cited within this disclosure, are incorporated by reference herein in their entirety into this disclosure:
1. Ojima, K., Shiraiwa, K., Soga, K. et al. Ligand-directed two-step labeling to quantify neuronal glutamate receptor trafficking. Nat. Commun. (2021) 12:831.
2. Muroski, M., Oh, E., Deschamps, J. R. et al. Gold-nanoparticle-mediated depolarization of membrane potential is dependent on concentration and tethering distance from the plasma membrane Part. Part. Syst. Charact. (2019) 36:1800493.
3. Sangtani, A., Nag. O. K, Oh, E., et al. Quantum dot-enabled membrane-tethering and enhanced photoactivation of chlorin-e6. J Nanopart Res (2021) 23:159.

In one exemplary embodiment, the present subject disclosure comprises a

vesicle for transporting phosphocreatine (PCr). The vesicle includes a fusogenic liposome containing phosphocreatine (PCr) within its core.

In another exemplary embodiment, the present subject disclosure comprises a method for delivering phosphocreatine (PCr) to cellular cytosol without direct interaction of the PCr with cellular plasma membrane. The method includes creating a fusogenic liposome containing phosphocreatine (PCr) within its core.

### BRIEF DESCRIPTION OF THE DRAWINGS

The patent or application file may contain at least one drawing executed in color. Copies of this patent or patent application publication with color drawing(s) will be provided by the Office upon request and payment of the necessary fee.

FIG. 1 depicts a phosphocreatine pathway, according to an exemplary embodiment of the present subject disclosure.

FIG. 2A shows the structures of phosphocreatine (PCr) and various phospholipids used for liposome synthesis and labeling, according to an exemplary embodiment of the present subject disclosure.

FIG. 2B depicts (left panel) liposomes containing PCr in the aqueous core fuse to the plasma membrane and deliver PCr to the cytosol to mediate production of ATP, while (right panel) naked PCr does not cross the plasma membrane, but rather interacts with the membrane bilayer and renders the plasma membrane more rigid, according to an exemplary embodiment of the present subject disclosure.

FIG. 3A depicts dynamic light scattering data showing the distribution of the hydrodynamic diameter of the various liposomal preparations, in this case DOPE/DOTAP/Rhod-PE/Calcein, according to an exemplary embodiment of the present subject disclosure.

FIG. 3B depicts dynamic light scattering data showing the distribution of the hydrodynamic diameter of the various liposomal preparations, in this case DOPE/DOTAP/NBD-PE/DAPI, according to an exemplary embodiment of the present subject disclosure.

FIG. 3C depicts dynamic light scattering data showing the distribution of the hydrodynamic diameter of the various liposomal preparations, in this case DOPE/DOTAP/NBD-PE/PCr, according to an exemplary embodiment of the present subject disclosure.

FIG. 3D depicts dynamic light scattering data showing the distribution of the hydrodynamic diameter of the various liposomal preparations, in this case DOPE/DOTAP/NBD-PE/PCr/DAPI, according to an exemplary embodiment of the present subject disclosure.

FIG. 3E shows a table summarizing the average diameter, polydispersity index (PDI), and zeta potential of the liposome formulations, according to an exemplary embodiment of the present subject disclosure.

FIG. 4A depicts HEK 293T/17 cells showing Rhod-PE (red) signal confirming fusion of the liposomal lipids with the plasma membrane coupled with intracellular delivery of the cell-impermeable fluorophore, calcein (green), according to an exemplary embodiment of the present subject disclosure.

FIG. 4B depicts, after liposome fusion, calcein was added to the extracellular medium where it accumulated over time in the extracellular spaces, demonstrating the integrity of the plasma membrane after liposome fusion, according to an exemplary embodiment of the present subject disclosure.

FIG. 4C shows quantification of the images in FIG. 4B showing the time-dependent extracellular accumulation of calcein (squares) while the intracellular calcein signal remains constant (circles), according to an exemplary embodiment of the present subject disclosure.

FIG. 5A depicts time-resolved imaging of HEK 293T/17 cells showing clear NBD-PE staining of the plasma membrane confirming successful liposome fusion and DAPI staining of nuclei demonstrating DAPI delivery to the cytosol and nucleus, according to an exemplary embodiment of the present subject disclosure.

FIG. 5B depicts quantification of cellular NBD-PE staining showing that while the NBD-PE staining plateaus at ˜15 min, the DAPI signal continues to increase over the 30 min imaging window, according to an exemplary embodiment of the present subject disclosure.

FIG. 5C depicts quantification of cellular DAPI staining showing that while the NBD-PE staining plateaus at ˜15 min, the DAPI signal continues to increase over the 30 min imaging window, according to an exemplary embodiment of the present subject disclosure.

FIG. 6A depicts PCr-loaded fusogenic liposomes mediate increased ATP production in HEK 293T/17 cells through a micrograph showing the increased red fluorescence of the ATP-Red probe in cells 30 min after incubation with DOPE/DOTAP/NBD-PE/PCr liposomes, according to an exemplary embodiment of the present subject disclosure.

FIG. 6B shows a time-resolved quantification of increased ATP production in PCr(+) and PCr(−) cells over 30 min period, according to an exemplary embodiment of the present subject disclosure.

FIG. 6C shows overall increase in ATP production in cells incubated with PCr(+) liposomes without (gray bar) and with (hatched bar) DAPI, according to an exemplary embodiment of the present subject disclosure.

FIG. 7 depicts cellular viability of HEK 293T/17 cells incubated with liposomal formulations, according to an exemplary embodiment of the present subject disclosure.

### DETAILED DESCRIPTION OF THE SUBJECT DISCLOSURE

The present subject disclosure addresses the shortcomings of conventional techniques, as discussed above.

### Definitions

Before describing the present subject disclosure in detail, it is to be understood that the terminology used in the specification is for the purpose of describing particular embodiments, and is not necessarily intended to be limiting. Although many methods, structures and materials similar, modified, or equivalent to those described herein can be used in the practice of the present subject disclosure without undue experimentation, the preferred methods, structures and materials are described herein. In describing and claiming the present subject disclosure, the following terminology will be used in accordance with the definitions set out below.

As used herein, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

### Overview

This subject disclosure describes in detail the use of fusognenic liposomes, loaded in their interior with PCr, to achieve the efficient cellular delivery of PCr to the cellular cytosol. Liposomes are a spherical form of nanoparticle (NP) comprised of a phospholipid bilayer encapsulating an aqueous core that is amenable for loading water-soluble cargos. Fusogenic liposomes are a special class of liposomes that are specifically tailored to fuse to the plasma membrane of target cells. This results in the connection and incorporation of liposomal phospholipids into the cell plasma membrane followed by the delivery of the liposomal contents into the cellular cytosol. There are examples of fusognenic liposomes for the delivery of proteins, nucleic acids, antimicrobial agents, and even nanoparticles intracellularly. [5-8] However, there are no reports of the direct intracellular delivery of PCr for the enhancement of ATP generation. The present subject disclosure details the development of fusogenic liposomal vehicles for the cytosolic delivery of PCr. This delivery strategy overcomes the limitations of PCr direct interaction with the plasma membrane lipids and subsequent entrapment within endosomal vesicles. Further, it delivers PCr to the cellular cytosol where it is able to easily access mitochondria.

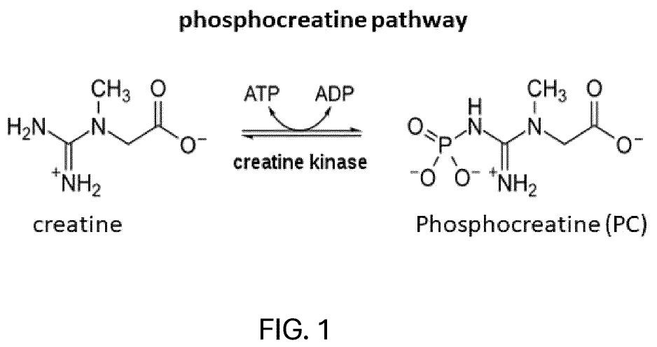

FIG. 1 depicts a phosphocreatine pathway. Phosphocreatine (PCr) is generated from the phosphorylation of creatine by the enzyme creatine kinase using ATP as the phosphate donor. Conversely, phosphocreatine serves as the phosphate donor to ADP to regenerate cellular ATP pools.

Reduction to practice is demonstrated by formulating a series of fusogenic liposomes composed primarily of DOPE, DOTAP, and dye-labeled lipids such as Rhod-PE or NBD-PE. These liposomal preparations were loaded with chromophores and different combinations of cell-impermeable molecules such as calcein, DAPI, and PCr, none of which internalize into the cell when from extracellular medium. We demonstrate liposome-mediated intracellular delivery of PCr using confocal microscopy imaging of live human embryonic kidney (HEK) 293T/17 cells. Successful delivery of PCr to the cellular cytosol was demonstrated by the simultaneous delivery of calcein and DAPI to the cytosol (using the fluorescent signal of calcein and DAPI to track delivery of the liposomal contents to the cellular cytosol). PCr delivery was further confirmed by the induction of increased ATP production in live HEK 293T/17 cells incubated with PCr-containing liposomes compared to non-PCr-loaded liposomes.

The following references, cited within this disclosure, are incorporated by reference herein in their entirety into this disclosure:
a) Synthesis of liquid crystal nanoparticles and PEG-Chol conjugation onto the surface

Nag, O. K., et al., Lipid Raft-Mediated Membrane Tethering and Delivery of Hydrophobic Cargos from Liquid Crystal-Based Nanocarriers. Bioconjugate Chem. 2016. 27(4): 982-993.
b) ZnPC and ZnPC loaded NPs for PDT Kim, J., et al., Selective photosensitizer delivery into plasma membrane for effective photodynamic therapy. J. Control Release, 2014. 191: 98-104.

Potential uses of the subject disclosure include, but are not limited to: (1) the use of PCr-loaded liposomes for augmented ATP generation in 2-D and 3-D tissue culture systems (cell-and organ-on-chip systems) and (2) use in vivo as nanoparticle-based therapeutics for cellular energy production. Many other uses are within the purview of the present subject disclosure as appreciated by one having ordinary skill in the art after consideration of the present subject disclosure. For sake of simplicity, the examples provided herein demonstrate an intracellular delivery of PCr through PCr-loaded liposomes, however, other substances may also be delivered through the techniques provided for herein.

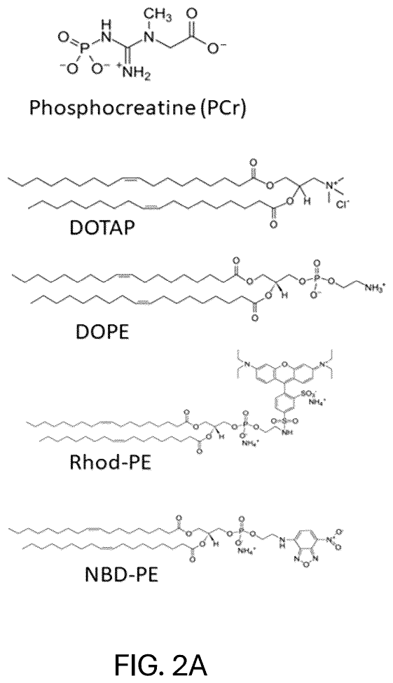

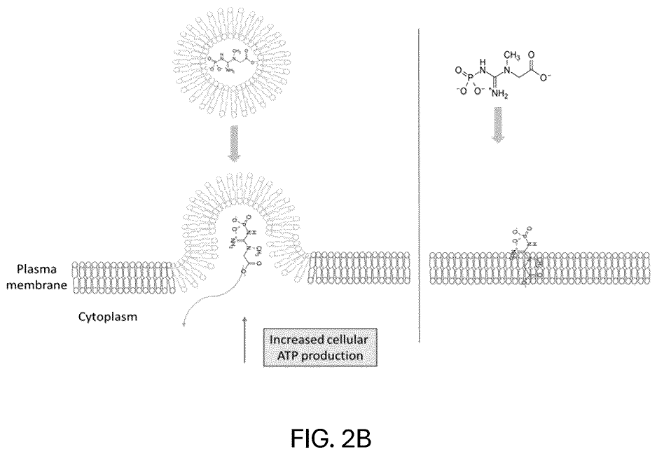

For successful liposome membrane-plasma membrane fusion, the fluidity of the liposomal lipid bilayer should exhibit inverted hexagonal structure in the lamellar phase. [9-11] This configuration helps to mediate the fusion of the liposomal membrane with the cell's plasma membrane. The formation of inverted hexagonal phase in the lamellar phase can be induced by incorporating ratiometric mixtures of cationic and nonionic lipids containing unsaturated fatty acid chains. Here, we used the cationic phospholipid 1,2-dioleoyl-3-trimethylammonium-propane (DOTAP) and the nonionic phospholipid 1,2-dioleoyl-sn-glycero-3-phosphoethanolamine (DOPE). FIG. 2 shows the basic premise of the liposomal formulation where ratiometric mixes of various phospholipids were used to controllably modulate the fluidity of the liposome membrane and to track the fusion of the liposome membrane with the cellular plasma membrane.

We formulated a series of fusogenic liposomes composed of DOTAP and DOPE mixed at a 1:1 ratio. Aromatic phospholipids bearing fluorescent dyes (e.g., 1,2-dioleoyl-sn-glycero-3-phosphoethanolamine-N-lissamine rhodamine B sulfonyl; (Rhod-PE) and 1,2-dipalmitoyl-sn-glycero-3-phosphoethanolamine-N-(7-nitro-2-1,3-benzoxadiazol-4-yl; (NBD-PE)) were included to track the efficiency of liposome fusion with the cell's plasma membrane. Further, as PCr is not inherently fluorescent, the liposomes were loaded in their aqueous core with either the cell-impermeable fluorescent DNA stain, DAPI (4′,6-diamidino-2-phenylindole) or the fluorophore calcein to track the delivery of liposomal contents to the interior of the cell.

FIG. 2 depicts a schematic of fusogenic liposomal delivery of phosphocreatine to cells. (FIG. 2A) Shown are the structures of phosphocreatine (PCr) and the phospholipids used for liposome synthesis and labeling. DOTAP and DOPE are ionic and nonionic nonsaturated lipids, respectively. The fluorescent lipids Rhod-PE and NBD-PE are used for tracking the fusion of liposomal membrane to the plasma membrane. Full phospholipid names are specified in the text. (FIG. 2B) Liposomes containing PCr in the aqueous core fuse to the plasma membrane and deliver PCr to the cytosol to mediate production of ATP (left panel). Naked PCr, due to its net negative charge, does not cross the plasma membrane (right panel). Rather, it interacts with the membrane bilayer and renders the plasma membrane more rigid.

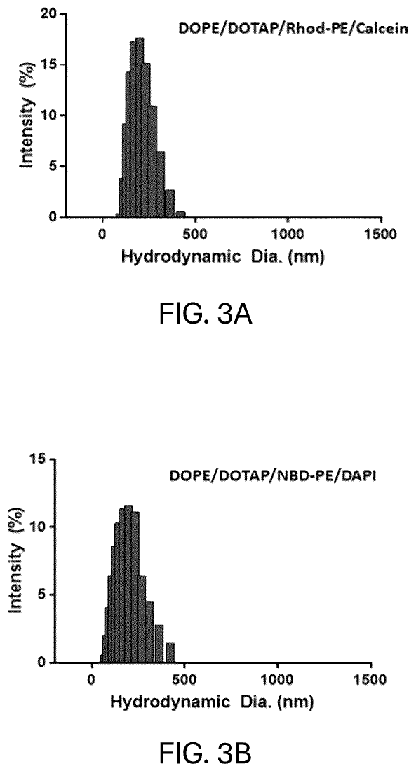

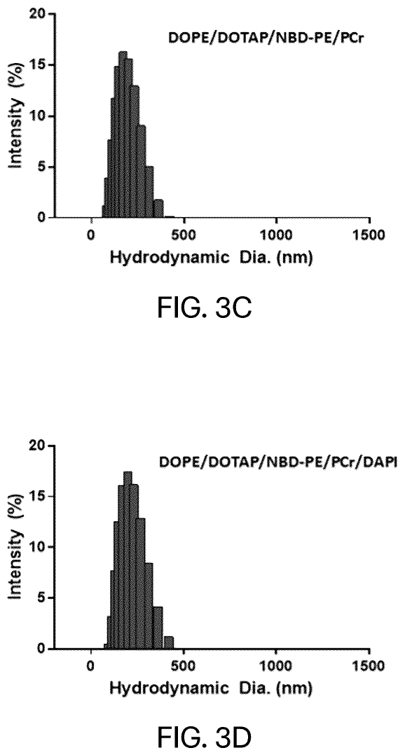

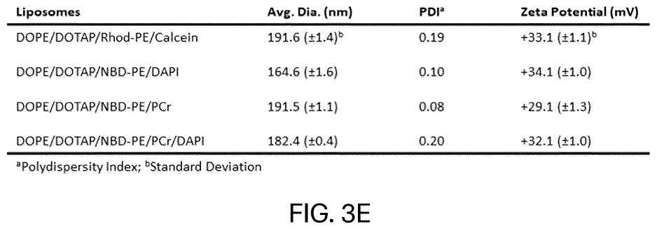

We prepared four different liposomal samples containing DOPE and DOTAP (1:1) as the main phospholipid composition and these were doped with an aromatic fluorescent lipid, either Rhod-PE (red fluorescence) or NBD-PE (green fluorescence) to facilitate both liposome: membrane fusion and for tracking their fusogenic capacity using confocal imaging. To test fusion and cytosolic delivery of the cargoes loaded inside, we prepared liposomes as follows: (1) DOPE/DOTAP/Rhod-PE (560 nm excitation (ex.)/ 583 nm emission (em.)) loaded with calcein (5 mM, 495 nm ex./ 515 nm em.) in the aqueous core (DOPE/DOTAP/Rhod-PE/Calcein), and DOPE/DOTAP/NBD-PE (460 nm ex/535 nm em) loaded with (2) DAPI (2 mM, 359 nm ex./457 nm em.) only, (3) loaded with PCr only, and (4) both DAPI and PCr. To achieve uniform size consistency among the different preparations, the liposomes were extruded through polylobate membranes (200 nm). We first characterized the NPs using dynamic light scattering to assess their hydrodynamic diameter and zeta potential measurements to determine their overall surface charge. FIG. 3 shows the results of these analyses. The data show that all of the liposomal preparations exhibited consistent hydrodynamic diameter (<200 nm) with a narrow polydispersity index (PDI) across the different preparations. The concentration of all of the liposomal preparations was determined to be ˜50-60 pM. The liposomes also showed highly positive (˜+30 mV) surface charges across the different preparations.

FIG. 3 depicts characterization of fusogenic liposomes for PCr delivery to cells. (FIG. 3A-D) Dynamic light scattering data showing the distribution of the hydrodynamic diameter of the various liposomal preparations. (FIG. 3E) Table summarizing the average diameter, polydispersity index (PDI), and zeta potential of the liposome formulations.

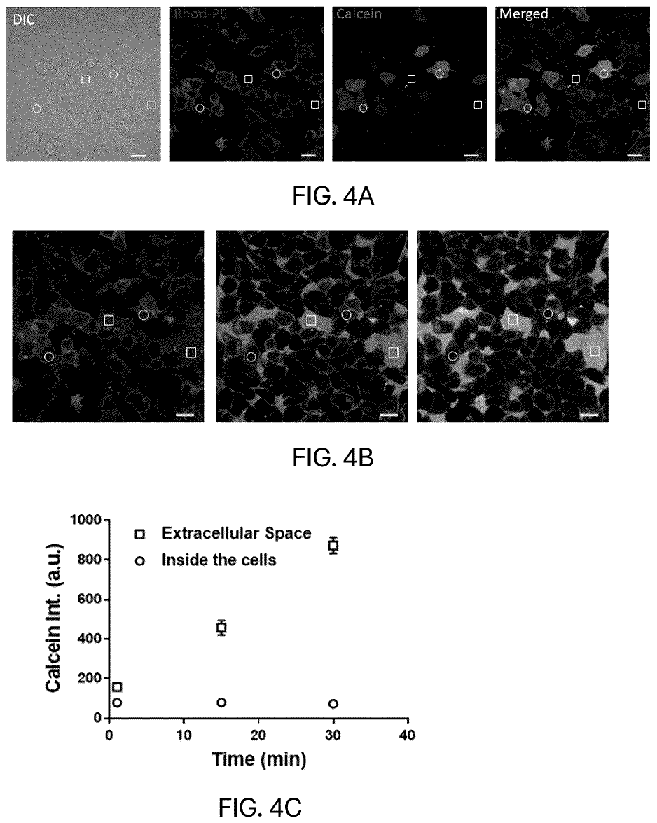

Next, we tested the liposomes for their fusogenic properties. Live human embryonic kidney cells (HEK 293T/17) were incubated with DOPE/DOTAP/Rhod-PE liposomes loaded with calcein, a cell-impermeable fluorophore, to determine the efficiency of liposomal fusion to the plasma membrane and the delivery of calcein to the cellular cytosol. We incubated the liposomes (3.0 pM) on the monolayer of HEK 293T/17 cells for 20 min at 37° C. followed by washing to remove unfused liposomes. The Rhod-PE and calcein signals were imaged and quantified using confocal fluorescence microscopy. FIG. 4A shows a representative confocal image where the Rhod-PE signal was clearly evident on the plasma membrane (in ˜50% of cells), indicating the fusion of the liposomal membrane with the plasma membrane. Further, in nearly all of these same cells, the calcein signal was clearly intracellular and homogeneous throughout the cell, indicating the successful delivery of the cell-impermeable calcein to the cytosol.

It was important to assess the potential toxicity or damage to plasma membrane caused by the positively charged lipid component, DOTAP which has been shown in some cases to create transient pores in the plasma membrane. [12,13] To test this we incubated free calcein (10 uM) on the cell monolayer which had already been incubated with the DOTAP-containing fusogenic liposomes and images were collected at 1, 15, and 30 min time points (FIG. 4B). Here, we observed that the intracellular green fluorescence signal remained unchanged while there was a steady, time-dependent increase in the green fluorescence signal in the extracellular spaces (FIG. 4C). Over the 30 min time period we observed an ˜6-fold increase in the extracellular green calcein signal compared to the intracellular calcein signal. These results confirm the successful fusion of the liposomes with the plasma membrane and delivery of calcein to the cell interior while at the same time there was no deleterious effects on plasma membrane integrity.

FIG. 4 depicts fusogenic liposomes and intracellular delivery of calcein. (FIG. 4A) HEK 293T/17 cells showing Rhod-PE (red) signal confirming fusion of the liposomal lipids with the plasma membrane coupled with intracellular delivery of the cell-impermeable fluorophore, calcein (green). (FIG. 4B) After liposome fusion, calcein was added to the extracellular medium where it accumulated over time in the extracellular spaces, demonstrating the integrity of the plasma membrane after liposome fusion. Scale bar is 20μm. (FIG. 4C) Quantification of the images in (FIG. 4B) showing the time-dependent extracellular accumulation of calcein (boxes) while the intracellular calcein signal remains constant (circles).

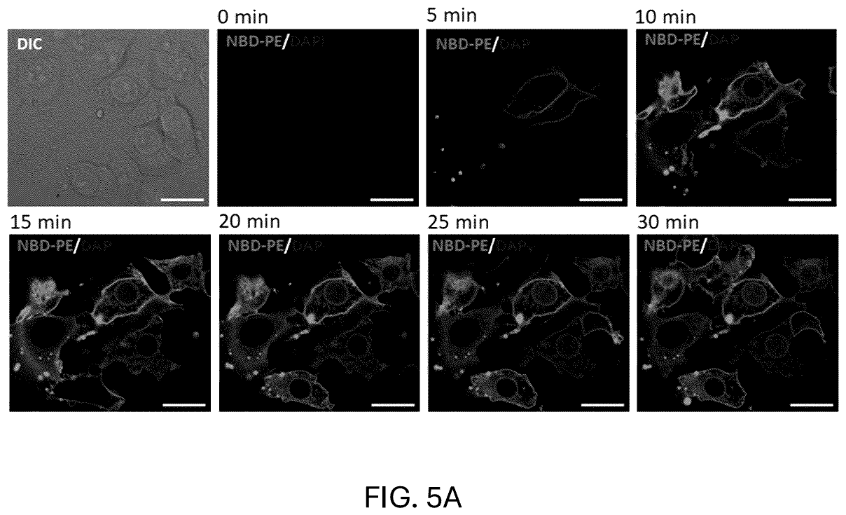

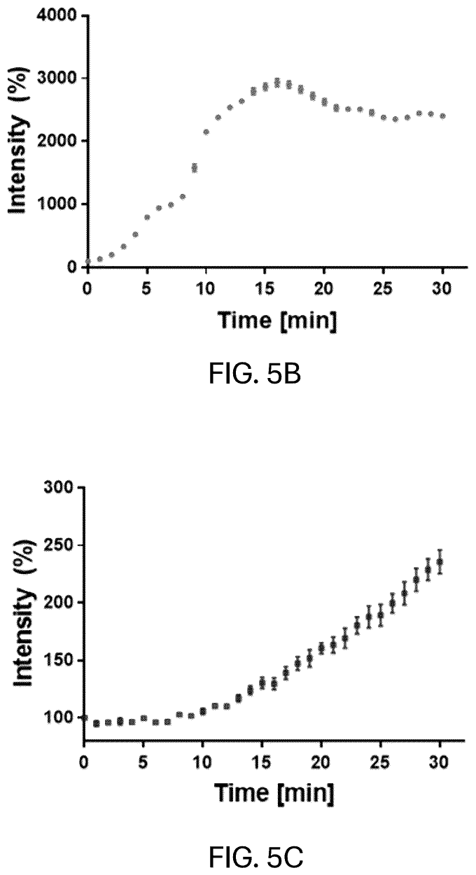

We further demonstrated the functionality and compatibility of the fusogenic liposomes with a phospholipid bearing a different aromatic dye (NBD-PE, green). In this instance, we prepared DOPE/DOTAP/NBD-PE loaded with 4′,6-diamidino-2-phenylindolenucleic acid (DAPI), a blue fluorescent DNA stain. Analogous to the calcein experiments, DAPI is cell-impermeable and DNA staining was used to confirm successful intracellular delivery. FIG. 5A shows that when incubated on HEK 293T/17 cells for 30 min the DAPI-loaded DOPE/DOTAP/NBD-PE liposomes showed both robust green staining of the plasma membrane (NBD) confirming fusion to the plasma membrane and a time-resolved increase in blue nuclear staining, confirming intracellular delivery of DAPI and binding to DNA in the nucleus. Time-resolved quantification of NBD-PE staining of the plasma membrane (FIG. 5B) and DAPI staining of the nuclei (FIG. 5C) showed that while maximum green NBD fluorescence staining of the plasma membrane was achieved at ˜15 min, the DAPI signal in the nucleus continued to increase over the 30 min imaging window. This is consistent with the continued influx of DAPI into the cytosol and eventually into the nucleus coupled with the increased fluorescence upon binding to the DNA. Overall, these results clearly demonstrated efficient delivery of DAPI from DOPE/DOTAP/NBD-PE to inside the cells.

FIG. 5 depicts (FIG. 5A) Time-resolved imaging of HEK 293T/17 cells showing clear NBD-PE staining of the plasma membrane confirming successful liposome fusion and DAPI staining of nuclei demonstrating DAPI delivery to the cytosol and nucleus. Quantification of cellular NBD-PE (FIG. 5B) and DAPI (FIG. 5C) staining showing that while the NBD-PE staining plateaus at ˜15 min, the DAPI signal continues to increase over the 30 min imaging window. Scale bar is 20 μm.

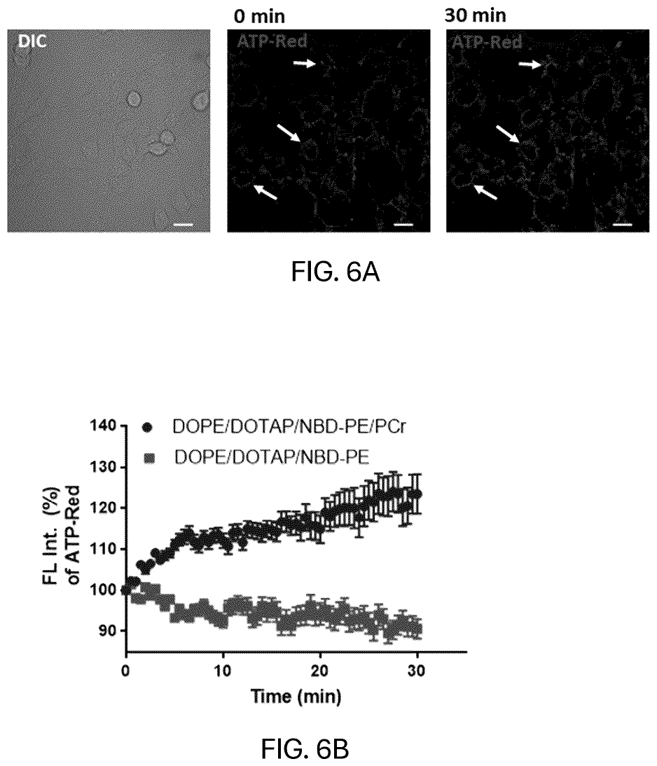

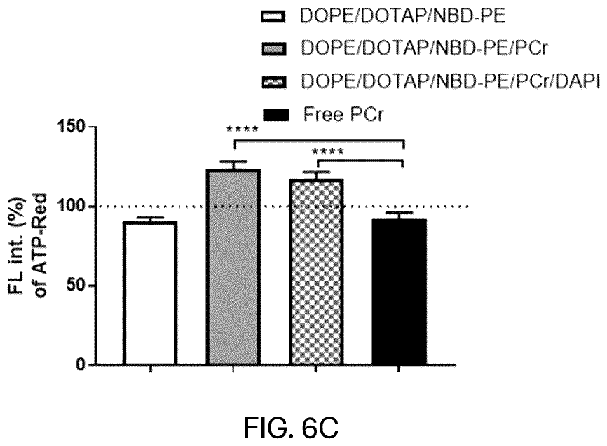

Having established the utility of the fusogenic liposome system, we next sought to demonstrate the cytosolic delivery of PCr and the concomitant increase in cellular ATP levels. To do this, we synthesized liposomes containing PCr (DOPE/DOTAP/NBD-PE/PCr) and those without PCr (DOPE/DOTAP/NBD-PE). FIG. 6A shows the time-resolved response of the fluorescent ATP-Red probe in HEK 293T/17 cells that were incubated with DOPE/DOTAP/NBD-PE/PCr liposomes. This probe increases in fluorescence in a quantitative manner upon binding to ATP [14]. The fluorescence micrographs show the increase in cellular fluorescence (red) 30 min after incubation of the cells with the PCr-containing liposomes. FIG. 6B shows the time-resolved quantitative comparison of fluorescence response in cells incubated with PCr-containing liposomes (blue trace) and cells incubated with cells incubated with non-PCr liposomes (red trace). We observed a 23% increase in ATP levels in PCr(+) cells over the 30 min window. While cells incubated with DOPE/DOTAP/NBD-PE or PCr only showed no measurable increase in ATP-Red signal, cells incubated with DOPE/DOTAP/NBD-PE/PCr and DOPE/DOTAP/NBD-PE/PCr/DAPI showed increases of 23% and 17%, respectively, over the entire 30 min observation period (FIG. 6C).

FIG. 6 depicts PCr-loaded fusogenic liposomes mediate increased ATP production in HEK 293T/17 cells. (FIG. A) Micrograph showing the increased red fluorescence of the ATP-Red probe in cells 30 min after incubation with DOPE/DOTAP/NBD-PE/PCr liposomes. The white arrows denote specific cells exhibiting increased fluorescence. Scale bar, 20 μm. (FIG. B) Time-resolved quantification of increased ATP production in PCr(+) and PCr(−) cells over 30 min period. (FIG. C) Overall increase in ATP production in cells incubated with PCr(+) liposomes without (gray bar) and with (hatched bar) DAPI. Controls were cells incubated with liposomes containing no PCr (white bar) and free PCr (black bar).

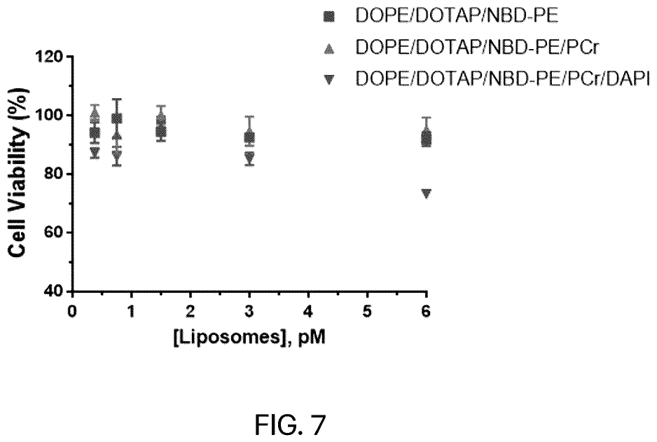

Finally, we determined the viability of HEK 293T/17 cells incubated with the various liposome formulation. Viability was assessed using a cellular proliferation assay based on a tetrazolium compound (MTS) that, in viable cells, is converted into a blue formazan product that absorbs at 590 nm. FIG. 7 shows the resulting cellular viability when cells were incubated for 30 min with liposomes up to concentration of 6 pM. DOPE/DOTAP/NBD-PE and DOPE/DOTAP/NBD-PE/PCr liposomes mediated cellular viabilities of >90% at 6 pM liposome concentration. Cells incubated with DOPE/DOTAP/NBD-PE/PCr liposomes showed viability of ˜75% at 6 pM. This is not surprising given the fact that the DAPI tracer binds to nuclear DNA and likely inhibits proliferation.

FIG. 7 depicts cellular viability of HEK 293T/17 cells incubated with liposomal formulations. Viability was determined using MTS colorimetric assay after liposomes were incubated on the cells (and subsequently removed) and the cells were allowed to proliferate for 72 h.

### Advantages and New Features

This subject disclosure demonstrates and encompasses key advantages and new features compared to the current state of the art, among others:
(1) The subject disclosure details the use of fusogenic liposomes of defined composition as an efficient carrier for delivery of drugs or dyes (e.g., calcein, DAPI) and phosphocreatine into living cells;
(2) Intracellular delivery of PCr offers various advantages, including 1) the protection of PCr from extracellular hydrolysis, 2) the minimization of PCr interaction with cellular membrane phospholipids, and 3) the direct delivery of PCr into the cytosol while avoiding other complication and competing internalization pathways, such as endocytosis that occur when using other types of delivery vehicles (e.g., non-fusogenic liposomes); and
(3) PCr delivered by fusogenic liposomes entering the cytosol while not significantly interacting with or lost to the cellular plasma membrane is a novel and nonobvious feature of the subject disclosure given the known tendency for PCr to interact with and be sequestered by the plasma membrane.

### Concluding Remarks

Although the present subject disclosure has been described in connection with preferred embodiments thereof, it will be appreciated by those skilled in the art that additions, deletions, modifications, and substitutions not specifically described may be made without departing from the spirit and scope of the subject disclosure. Terminology used herein should not be construed as being “means-plus-function” language unless the term “means” is expressly used in association therewith.

The foregoing disclosure of the exemplary embodiments of the present subject disclosure has been presented for purposes of illustration and description. It is not intended to be exhaustive or to limit the subject disclosure to the precise forms disclosed. Many variations and modifications of the embodiments described herein will be apparent to one of ordinary skill in the art in light of the above disclosure. The scope of the subject disclosure is to be defined only by the claims appended hereto, and by their equivalents.

Further, in describing representative embodiments of the present subject disclosure, the specification may have presented the method and/or process of the present subject disclosure as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present subject disclosure should not be limited to the performance of their steps in the order written, and one skilled in the art can readily appreciate that the sequences may be varied and still remain within the spirit and scope of the present subject disclosure.

All documents mentioned herein are hereby incorporated by reference for the purpose of disclosing and describing the particular materials and methodologies for which the document was cited.

The following references, cited within this disclosure, are incorporated by reference herein in their entirety into this disclosure:
1. Guimarães-Ferreira, L. Role of the phosphocreatine system on energetic homeostasis in skeletal and cardiac muscles. Einstein (Sao Paulo) 2014, 12, 126-131, doi:10.1590/s1679-45082014rb2741.
2. Liu, W.; Qaed, E.; Zhu, H. G.; Dong, M. X.; Tang, Z. Non-energy mechanism of phosphocreatine on the protection of cell survival. Biomedicine & Pharmacotherapy 2021, 141, 111839, doi:https://doi.org/10.1016/j.biopha.2021.111839.
3. Lourdes Guerrero-Ontiveros, M.; Wallimann, T. Creatine supplementation in health and disease. Effects of chronic creatine ingestion in vivo: Down-regulation of the expression of creatine transporter isoforms in skeletal muscle. Molecular and Cellular Biochemistry 1998, 184, 427-437, doi:10.1023/A:1006895414925.
4. Mercimek-Andrews, S.; Salomons, G. S. Creatine Deficiency Disorders. In GeneReviews(®), Adam, M. P., Mirzaa, G. M., Pagon, R. A., Wallace, S. E., Bean, L. J. H., Gripp, K. W., Amemiya, A., Eds.; © 1993-2023, University of Washington, Seattle. GeneReviews is a registered trademark of the University of Washington, Seattle: Seattle WA, 1993.
5. Kube, S.; Hersch, N.; Naumovska, E.; Gensch, T.; Hendriks, J.; Franzen, A.; Landvogt, L.; Siebrasse, J.-P.; Kubitscheck, U.; Hoffmann, B.; et al. Fusogenic Liposomes as Nanocarriers for the Delivery of Intracellular Proteins. Langmuir: the ACS journal of surfaces and colloids 2017, 33, 1051-1059, doi:10.1021/acs.langmuir.6b04304.
6. Csiszár, A.; Csiszar, A. ; Pinto, J. T.; Gautam, T.; Kleusch, C.; Hoffmann, B.; Tucsek, Z.; Toth, P.; Sonntag, W. E.; Ungvari, Z. Resveratrol encapsulated in novel fusogenic liposomes activates Nrf2 and attenuates oxidative stress in cerebromicrovascular endothelial cells from aged rats. J Gerontol A Biol Sci Med Sci 2015, 70, 303-313, doi:10.1093/gerona/glu029.
7. Kleusch, C.; Hersch, N.; Hoffmann, B.; Merkel, R.; Csiszăr, A. Fluorescent lipids: functional parts of fusogenic liposomes and tools for cell membrane labeling and visualization. Molecules 2012, 17, 1055-1073, doi:10.3390/molecules17011055.
8. Moch, M.; Windoffer, R.; Schwarz, N.; Pohl, R.; Omenzetter, A.; Schnakenberg, U.; Herb, F.; Chaisaowong, K.; Merhof, D.; Ramms, L.; et al. Effects of Plectin Depletion on Keratin Network Dynamics and Organization. PloS one 2016, 11, e0149106, doi:10.1371/journal.pone.0149106.
9. Marrink, S. J.; Tieleman, D. P. Molecular dynamics simulation of spontaneous membrane fusion during a cubic-hexagonal phase transition. Biophysical journal 2002, 83, 2386-2392, doi:10.1016/s0006-3495(02)75252-1.
10. Chernomordik, L. V.; Kozlov, M. M. Mechanics of membrane fusion. Nat Struct Mol Biol 2008, 15, 675-683, doi:10.1038/nsmb.1455.
11. Siegel, D. P.; Epand, R. M. The mechanism of lamellar-to-inverted hexagonal phase transitions in phosphatidylethanolamine: implications for membrane fusion mechanisms. Biophysical journal 1997, 73, 3089-3111, doi:10.1016/s0006-3495(97)78336-x.
12. Hersch, N.; Wolters, B.; Ungvari, Z.; Gautam, T.; Deshpande, D.; Merkel, R.; Csiszar, A.; Hoffmann, B.; Csiszár, A. Biotin-conjugated fusogenic liposomes for high-quality cell purification. Journal of Biomaterials Applications 2016, 30, 846-856, doi:10.1177/0885328215603026.
13. Naumovska, E.; Ludwanowski, S.; Hersch, N.; Braun, T.; Merkel, R.; Hoffmann, B.; Csiszár, A. Plasma membrane functionalization using highly fusogenic immune activator liposomes. Acta biomaterialia 2014, 10, 1403-1411, doi:10.1016/j.actbio.2013.12.009.
14. Wang, L.; Yuan, L.; Zeng, X.; Peng, J.; Ni, Y.; Er, J. C.; Xu, W.; Agrawalla, B. K.; Su, D.; Kim, B.; et al. A Multisite-Binding Switchable Fluorescent Probe for Monitoring Mitochondrial ATP Level Fluctuation in Live Cells. Angewandte Chemie International Edition 2016, 55, 1773-1776, doi:https://doi.org/10.1002/anie.201510003.

What is claimed is:

1. A vesicle for transporting phosphocreatine (PCr), comprising:
a fusogenic liposome containing phosphocreatine (PCr) within its core.

2. The vesicle for transporting phosphocreatine of claim 1, wherein the fusogenic liposome comprises:
a mixture of phospholipids 1,2-dioleoyl-sn-glycero-3-phosphoethanolamine (DOPE), and 1,2-dioleoyl-3-trimethylammonium-propane, chloride salt (DOTAP).

3. The vesicle for transporting phosphocreatine of claim 2, wherein the mixture of DOPE to DOTAP has a ratio of 1:1.

4. The vesicle for transporting phosphocreatine of claim 2, wherein the fusogenic liposome further comprises:
aromatic lipids 1,2-dioleoyl-sn-glycero-3-phosphoethanolamine-N-[7-nitro-2-1,3-benzoxadiazol-4-yl (NBD-PE), and 1,2-dioleoyl-sn-glycero-3-phosphoethanolamine-N-[lissamine Rhodamine B sulfonyl] (Rhod-PE) to fluorescently track and image fusion of the fusogenic liposome to a mammalian plasma membrane.

5. A method for delivering phosphocreatine (PCr) to cellular cytosol without direct interaction of the PCr with cellular plasma membrane, comprising:
creating a fusogenic liposome containing phosphocreatine (PCr) within its core.

6. The method of claim 5, wherein the fusogenic liposome comprises:
a mixture of phospholipids 1,2-dioleoyl-sn-glycero-3-phosphoethanolamine (DOPE), and 1,2-dioleoyl-3-trimethylammonium-propane, chloride salt (DOTAP).

7. The method of claim 6, wherein the mixture of DOPE to DOTAP has a ratio of 1:1.

8. The method of claim 6, wherein the fusogenic liposome further comprises:
aromatic lipids 1,2-dioleoyl-sn-glycero-3-phosphoethanolamine-N-[7-nitro-2-1,3-benzoxadiazol-4-yl (NBD-PE), and 1,2-dioleoyl-sn-glycero-3-phosphoethanolamine-N-[lissamine Rhodamine B sulfonyl] (Rhod-PE) to fluorescently track and image fusion of the fusogenic liposome to a mammalian plasma membrane.
